(19) United States
(12) Patent Application Publication
(10) Pub. No.: US 2004/0030199 A1
(43) Pub. Date: Feb. 12, 2004

(54) Process for reducing alpha-haloketones to secondary alpha-haloalcohols

(76) Inventors: Robert R. Maughon, Midland, MI (US); George J. Frycek, Midland, MI (US); Paul C. Vosejpka, Midland, MI (US); Tim L. Allen, Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/060,626

(22) Filed: Jan. 29, 2002

(51) Int. Cl. C07C 31/34

(52) U.S. Cl. 568/842

(57) ABSTRACT

An α-haloalcohol is made by hydrogenating an α-haloketone with a hydrogenating agent in the presence of a transition metal-containing heterogeneous catalyst, under conditions such that an α-haloalcohol is formed. The reaction is particularly useful, for example, in a process to make epoxides which may be generally prepared by: (a) reducing an α-haloketone with a hydrogenating agent to form an α-haloalcohol; and (b) cyclizing the α-haloalcohol with base to make an epoxide.

### FIELD OF THE INVENTION

The present invention relates to a process for making α-haloalcohols from α-haloketones. More specifically, the present invention relates to a process for synthesizing α-haloalcohols through the hydrogenation of α-haloketones. The α-haloalcohols prepared by the process of the present invention are useful in a process for preparing epoxides.

### BACKGROUND OF THE INVENTION

There are many ketones known in the prior art and the hydrogenation of ketones is not uncommon. However, α-haloketones, because of the presence of unstable halogen-carbon bonds, are much more reactive than other ketones and one skilled in the art has found it very difficult to selectively hydrogenate α-haloketones to α-haloalcohols. There is still a long felt need for an economical and efficient process for the hydrogeneation of α-haloketones.

α-Haloketones known in the prior art can be converted to α-haloalcohols by various known processes. For example, several references disclose the reduction of α-haloketones using homogeneous catalyst systems. For example, U.S. Pat. No. 4,024,193 discloses a homogeneous hydrogenation process using the active form of a ruthenium triphenyl phosphine represented by the following formula: RuHCl(PR₃)₃, for the reduction of activated carbonyl compounds including α-chloroketones such as 1,3-dichloroacetone and α-chloroacetophenone. No yields or selectivities for the reactions are presented in U.S. Pat. No. 4,024,193.

Japanese Patent No. 63-297333 discloses a process for preparing 1,3-dichloro-2-propanol from 1,3-dichloroacetone using aluminum isopropoxide as a homogeneous catalyst with excess isopropanol as the hydrogen transfer reagent. Selectivities of 95% or less can be obtained using the process of Japanese Patent No. 63-297333, but no amount less than 0.01 equivalent of aluminum isopropoxide can be used.

Japanese Patent No. 09-104648 discloses a process for the hydrogenation of α-haloketones including α-chloroketones such as 1,3-dichloro-acetone to form 1,3-dichloro-2-propanol using a homogeneous ruthenium complex bearing a cyclopentadienone ligand. Using the process of Japanese Patent No. 09-104648, selectivities of 91%-98% of 1,3-dichloro-2-propanol are achieved; and less than 10,000 turnovers are demonstrated through sequential batchwise addition of the of α-haloketone.

WO 9800375 A1 and EP 295890 A2 describe processes for the asymmetric hydrogenation of α-haloketones such as chloroacetone to produce chiral alcohols using homogeneous ruthenium, iridium, rhodium, rhenium, cobalt, nickel, platinum, and palladium complexes bearing chiral ligands.

It is also well known to use α-haloalcohols for synthesizing epoxides. For instance, aforementioned Japanese Patent No. 09-104648 and Japanese Patent No. 63-297333 disclose a process to make epichlorohydrin by a three-step process as follows:

(1) α-chlorinating acetone with molecular chlorine in the presence of an iodine-containing promoter and lithium chloride to give 1,3-dichloroacetone;

(2) hydrogenating 1,3-dichloroacetone in the presence of a homogeneous catalyst to form 1,3-dichloro-2-propanol; and

(3) cyclizing 1,3-dichloropropanol with a base to make epichlorohydrin.

The limitation in all of the above known processes is the requirement of a homogeneous catalyst to accomplish the reduction of the α-haloketones selectively. The application of a homogeneous catalyst in the above processes limits the modes of reactor operation; and catalyst separation and reuse is non-trivial. It is, therefore, desirable to provide a heterogeneous catalyst which can accomplish the reduction of an α-haloketone with selectivities comparable to reduction of α-haloketone with homogeneous catalysts but which has the added advantage of being in a supported, heterogeneous form. A heterogenuous catalyst would advantegously allow for operation of a reduction process using the catalyst without the need for catalyst separation.

It is, therefore, an object of the present invention to provide a commercially feasible and easily controllable process for the effective reduction of an α-haloketone to form an α-haloalcohol using a heterogeneous catalyst.

Another object of the present invention is to provide an improved hydrogenation process for preparing α-haloalcohols from α-haloketones using a heterogeneous catalyst.

A further object of the present invention is to provide such a process which employs pressures and temperatures that allows it to be readily operated in a more economical manner.

Other objects of the present invention are to advance the art; and still other objects will appear hereinafter.

### SUMMARY OF THE INVENTION

One aspect of the present invention is directed to a process to make an α-haloalcohol comprising the step of reacting an α-haloketone with a hydrogenating agent, such as elemental hydrogen, in the presence of a heterogeneous transition metal-containing catalyst, under conditions such that an α-haloalcohol is formed.

The process of the present invention can be represented by the following general equation:

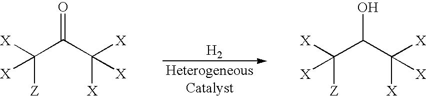

A second aspect of the present invention is directed to a process to make epoxides comprising the steps of:

(1) hydrogenating an α-haloketone to form an α-haloalcohol as described in the first aspect of the present invention; and

(2) cyclizing the α-haloalcohols to make epoxides.

The process of the present invention utilizes a heterogeneous catalyst, thus simplifying the mode of reactor operation and facilitating catalyst separation/re-use.

One key aspect of the present invention is the discovery of a heterogeneous catalyst which accomplishes this hydrogenation selectively. The process of the present invention is also useful in a process for synthesizing epoxides from α-haloalcohols.

The α-haloalcohol can now be conveniently and effectively prepared by an industrially advantageous process and from easily available materials.

The catalyst used in the process of the present invention is solid, and therefore, is easily recovered from the reaction mixture and is easily removed from the product.

### DETAILED DESCRIPTION OF THE INVENTION

The process of the present invention makes α-haloalcohols from α-haloketones. α-Haloketones of the present invention are represented by Formula I as follows:

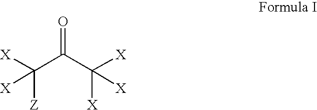

wherein each “X” is independently a halogen atom excluding fluorine, a hydrogen atom, or an organic group; and “Z” is a halogen atom excluding fluorine. 1,3-Dichloroacetone is one example of an α-haloketone of Formula I.

α-Haloalcohols of the present invention are represented by Formula II as follows:

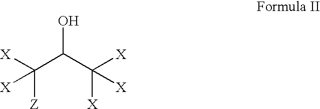

wherein each “X” is independently a halogen atom excluding fluorine, a hydrogen atom, or an organic group; and “Z” is a halogen atom excluding fluorine. 1,3-Dichloro-2-propanol is one example of an α-haloalcohol of Formula II.

Examples of suitable α-haloketones useful in the present invention include: 1-chloroacetone, 1,3-dichloroacetone; 1,3-dibromoacetone; 1,1,3-trichloroacetone, and mixtures thereof. The α-haloketones used in the present invention are most preferably unsubstituted 1,3-dihaloacetone to form 1,3-dihalo-2-propanol and 1-haloacetone to form 1-halo-2-propanol. 1,3-Dihaloacetone is represented by:

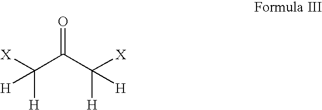

wherein each “X” is independently a halogen excluding fluorines. The “X” in Formula III above is preferably iodine, chlorine, or bromine; and most preferably chlorine.

The α-haloketone is hydrogenated by reaction with a hydrogenating agent. The hydrogenating agent useful in the present invention may be, for example, molecular hydrogen, an alcohol, or a combination thereof. The hydrogenating agent is preferably molecular hydrogen. Examples of suitable alcohols useful in the present invention can be primary or secondary alcohols such as methanol, ethanol and C₃-C₁₀ primary and secondary alcohols. Preferably, the alcohol is methanol. Examples of other secondary alcohols useful in the present invention are described in U.S. Pat. No. 2,860,146 which is incorporated herein by reference.

The reaction according to the present invention consumes one mole of hydrogenating agent per mole of α-haloalcohol which is made. Generally, at least about 0.6 moles of hydrogenating agent per mole of α-haloketone are available to be consumed during the course of the reaction, preferably at least about 0.75 moles of hydrogenating agent per mole of α-haloketone are available to be consumed during the course of the reaction, more preferably at least about 0.9 moles and most preferably at least about 1 mole are available to be consumed during the course of the reaction. When less than 1 mole of hydrogenating agent per mole α-haloketone is available to be consumed during the course of the reaction, complete conversion to the α-haloalcohol may not be obtained. However, not all of the hydrogenating agent need be available at the start of the reaction. The hydrogenating agent may be added step-wise or continuously as the reaction progresses. In this case, the reaction mixture at any one time may contain a stoichiometric excess of α-haloketone over hydrogenating agent. As one embodiment of the present invention, an excess of hydrogenating agent required may be used to complete the conversion of α-haloketone to α-haloketone to α-haloalcohol during the reaction. Generally, for example, from 10 percent to 20 percent excess hydrogenating agent may be used.

The maximum quantity of hydrogenating agent source is not critical and is governed by practical considerations such as pressure, reactor efficiency, and safety. When the hydrogenating agent source is gaseous, then the quantity of hydrogenating agent is preferably at least enough to provide the desired pressure. However, in most cases, the reactor preferably contains no more than about 1,000 moles of molecular hydrogen per mole of α-haloketone and more preferably contains no more than about 100 moles of molecular hydrogen per mole of α-haloketone. Gaseous hydrogenating agent sources, such as molecular hydrogen, are preferably used according to known methods for mixing a gaseous reagent with a liquid reaction mixture, such as bubbling the gas through the mixture with agitation or solubilizing the hydrogen under pressure.

The reaction of the present invention takes place in the presence of a heterogeneous transition metal-containing catalyst. The transition metal useful in the heterogeneous catalyst of the present invention may be one or more metals selected from any of Groups IB, IIB or IIIA-VIIIA on the periodic table of elements, as currently adopted by the International Union of Pure and Applied Chemistry (IUPAC), which is incorporated herein by reference. The catalyst metals useful in the present invention are selected such that under reaction conditions the metals catalyze the hydrogenation of substantially all of the carbonyl moieties on the α-haloketone molecule to alcohol moieties without substantially affecting the halogens which are bonded to the α-haloketone molecule. The catalyst metal is preferably selected from Group VIIIA of the IUPAC periodic table, including for example, iron, cobalt, nickel, ruthenium, rhodium, palladium, osmium, iridium, platinum and mixtures thereof. The catalyst metal is more preferably selected from the group consisting of ruthenium, iridium, rhodium, palladium, platinum, or mixtures thereof. The catalyst metal is most preferably selected from the group consisting of ruthenium, iridium or mixtures thereof.

An illustration of the catalyst of the present invention may be, for example, the iridium/ruthenium mixed metal catalysts disclosed in published European Patent Application 1140751, incorporated herein by reference. The atomic ratio of iridium metal to ruthenium metal in the catalyst is generally from about 0.02 to about 15, preferably from about 0.05 to about 10, more preferably from about 0.15 to about 8, and most preferably from about 0.3 to about 2.0.

The heterogeneous catalysts useful in the present invention may be, for example, a transition metal deposited or absorbed on an insoluble support such as silica, silylated silica, carbon, alumina, titania, zirconia, magnesia and other common supports known in the art as described in Poncelet et al. editors, Preparation of Catalysts III, New York, 1983; P. N. Rylander, Hydrogenation Methods, Academic Press, London, 1985; P. N. Rylander, Catalytic Hydrogenation Over Platinum Metals, Academic Press, New York, 1967; P. Rylander, Catalytic Hydrogenation in Organic Syntheses, Academic Press, New York, 1979 all which are incorporated herein by reference. The heterogeneous catalyst of the present invention may also be a transition metal coordinated to ligands bonded to a resin, for example ruthenium on phosphinated polystyrene. The catalyst is typically in the form of granules or pellets. The amount of active catalyst on a support is generally from about 0.1 percent (%) to about 25% and preferably from about 0.5 percent to about 15 percent.

One advantage of using a heterogeneous catalyst in the process of the present invention is the ability to separate the catalyst from the reaction solution by various means such as by filtration.

The ideal ratio of catalyst to reagents used in the present process varies depending upon flow rate, bed size, temperature, desired conversion, reagents and other factors of the present process. Usually, a heterogeneous catalyst bed contains about 0.0001 mole to about 100 moles of catalyst metal for each mole of α-haloketone which passes through the bed per hour.

The reaction of the present invention is optionally, but preferably, carried out in the presence of a solvent. The solvent used is preferably inert with respect to all of the reagents under the reaction conditions. The solvent may be selected such that: (1) the solvent does not boil under reaction conditions; and (2) the α-haloalcohol can be recovered from the solvent, for example by distillation, extraction, or any other known recovery means.

Examples of suitable solvents useful in the present invention include aromatic and aliphatic hydrocarbons, chlorinated hydrocarbons, ethers, glymes, glycol ethers, esters, alcohols, amides, and mixtures thereof. Specific examples of the solvents useful in the present invention include toluene, cyclohexane, hexane, methylene chloride, dioxane, dimethyl ether, diglyme, 1,2-dimethoxyethane, ethyl acetate, methanol, NMP, and mixtures thereof. The quantity of solvent used in the present invention is not critical and is governed primarily by practical considerations, such as the efficiency of the reactor. Generally, the amount of the solvent present in the reaction mixture ranges from 0 to about 99.99 weight percent.

In most preferred cases, the reaction mixture of the present invention preferably contains at least about 5 weight percent α-haloketone, more preferably at least about 10 weight percent, and most preferably at least about 20 weight percent. The reaction mixture can be neat (i.e. the reaction mixture can contain essentially 100 weight percent α-haloketone), but if a solvent is used in addition to the α-haloketone, the reaction mixture preferably contains no more than about 90 weight percent α-haloketone and more preferably no more than about 80 weight percent α-haloketone.

When the reaction mixture contains an alcohol, the reaction is preferably carried out under conditions which are substantially free of strong mineral acids such as hydrogen chloride, which may cause a reduction in selectivity and yields. “Substantially free” of strong mineral acids means that the concentration of such acids is low enough that the acids do not catalyze the formation of significant yields of ketals from the α-haloketone and alcohol. For example, the level of ketals formed by the acid catalyzed reaction between α-haloketone and an alcohol in the reaction mixture may be generally less than about 50 weight percent, preferably less than about 20 weight percent and most preferably less than about 1 percent.

Without intending to be bound to a particular theory, it is theorized that a strong acid catalyzes the reaction of α-haloketone and alcohol to form an undesirable ketal. In instances where the reaction mixture contains minor quantities of hydrogen halide, it is preferable to carry out the reaction in the presence of an acid scavenger if alcohol is present in the reaction mixture to prevent ketal formation.

Examples of suitable acid scavengers useful in the present invention include: alkali metal carbonates; alkali metal bicarbonates; alkali metal carboxylates; ammonium and phosponium carboxylates, bicarbonates, and carbonates; epoxides and mixtures thereof. Specific examples of acid scavengers include sodium carbonate, sodium bicarbonate, ammonium bicarbonate, ethylene oxide, propylene oxide, butylene oxide, epichlorohydrin and mixtures thereof. Epichlorohydrin is the preferred epoxide to serve as an acid scavenger.

The temperature of the reaction is not critical, provided that all of the reagents and catalyst are in intimate contact with each other. However, low temperatures require longer reaction times. The reaction temperature is preferably at least about −10° C., more preferably at least about 20° C. and most preferably at least about 50° C. The reaction temperature is preferably less than about 250° C., more preferably no more than about 150° C. and most preferably no more than about 120° C. The reaction temperature is preferably from about 0° C. to about 200° C. and more preferably from about 50° C. to about 120° C.

The reaction pressure is not critical as long as there is sufficient hydrogenating agent such as hydrogen to run the reaction in the reaction mixture. The pressure is preferably at least about 14 pound per square inch absolute (psia) (97 kilo Pascals (kPa), 1 atmosphere) and more preferably at least about 50 psia (340 kPa, 3.4 atmospheres). The pressure is preferably no more than about 3,000 psia (21 MPa, 204 atmospheres). Higher pressures could lead to shorter reaction times.

Usually the contact time in the hydrogenation reaction according to the present invention is less than about 72,000 seconds and preferably from about 36,000 seconds to about 180 seconds sufficient to achieve nearly theoretical conversion of 1 gram of α-haloketone to the α-haloalcohol per gram of catalyst.

Under the preferred process conditions, the α-haloketones is catalytically reacted with an excess of hydrogen at a temperature of from about 0° C. to about 200° C. for a contact time of from about 36,000 seconds to about 180 seconds for 1 gram of the α-haloketone per gram of catalyst, followed by recovery of the desired reaction product.

The product of the reaction of the present invention is an α-haloalcohol with a structure derived from the α-haloketone. The product may be recovered by known methods, such as extraction or distillation. The product may be recovered in yields as low as 2 percent, however, for economical purposes the product of the present invention is generally recovered in at least about 60 percent yields (based upon the initial quantity of α-haloketone), and preferably recovered in at least about 80 percent yields, more preferably in at least about 90 percent yields and most preferably in at least about 95 percent yields.

The α-haloalcohol prepared by the process of the present invention is an important intermediate reaction product. The reaction process of the present invention for making an α-haloalcohol is a particularly useful step in an overall process for making epoxides. Once the α-haloalcohol is made using the reduction/hydrogenation reaction of the present invention, the α-haloalcohol may be cyclized to make an epoxide by processes well-known in the art. For example, α-haloalcohols are useful in the preparation of epoxides through treatment of the α-haloalcohol with a base. Therefore, the present invention is useful in a process to synthesize epoxides, such as epichlorohydrin and propylene oxide, for example through a general process including the following step of:

(1) Hydrogenating an α-haloketone to form an α-haloalcohol; and

(2) Cyclizing of the α-haloalcohol with a base to produce an epoxide.

In another embodiment for preparing an epoxide, the α-haloketone may be formed by halogenating a ketone to product the α-haloketone prior to the hydrogenation step.

A key step in the process of the present invention is the selective hydrogenation of the α-haloketone to the α-haloalcohol such that the carbon-chlorine bonds of the α-haloketone remain intact during hydrogenation.

More particularly, the reaction process of the present invention may be used as one of the steps in a process to make, for example epihalohydrin or propylene oxide from acetone. As an illustration of the present invention, for example, the process to make epihalohydrin is detailed as follows:

In a Step (1) of the process of making epiholohydrin, acetone is halogenated to make 1,3-dihaloacetone. This step for the preparation of 1,3-dichloroacetone is described in, for example, U.S. Pat. No. 4,251,467 and JP 9255615 which are incorporated herein by reference.

In a Step (2) of the reaction process of the present invention, the 1,3-dihaloacetone is hydrogenated to form 1,3-dihalo-2-propanol. The preferred embodiments of this Step (2) are previously described above in this application. For example, one embodiment of the process of the present invention comprises the step of contacting a 1,3-dihaloacetone with at least a stoichiometric quantity of molecular hydrogen in the presence of a ruthenium-containing, iridium-containing or a ruthenium-iridium mixed metal containing catalyst and an aprotic solvent such as dioxane to produce 1,3-dihalo-2-propanol.

In a Step (3) of the present process, the 1,3-dihalo-2-propanol is converted to epihalohydrin. This Step (3) is well-known in the art of manufacturing epihalohydrin. The reaction of Step (3) is usually carried out by contacting the 1,3-dihalo-2-propanol with a strong base, such as an aqueous alkali metal hydroxide, including for example sodium hydroxide. Examples of the Step (3) reaction are described in U.S. Pat. No. 2,860,146 and in Australian Patent No. 630,238, which are incorporated herein by reference.

Epoxide processes which use the present invention may contain any one or more of the Steps (1), (2) and (3) described above, in addition to Step (2). The epoxide processes preferably contain Steps (1) and (2), more preferably contain Steps (1), (2) and (3).

In the processes to make epoxides such as epihalohydrin or propylene oxide, it is possible to start with a mixture containing an α-haloketone with other ketones, isomers, or reagents. In such cases, it is preferred that the α-haloketone of choice such as 1,3-dihaloacetone for making epihalohydrin or such as 1-haloacetone for making propylene oxide be the predominant ketone and that the product formed be predominantly epihalohydrin or propylene oxide, respectively. Because it is possible that the resultant product could be a mixture of epihalohydrin and propylene oxide, it is preferred to control the amounts of α-haloketones used in the processes to provide a desired product in a substantial amount. By “predominantly” herein it is meant that the desired product is present in greater than 50% by weight or more in a mixture of two major α-haloketone components and greater than 40% by weight or more in a mixture of three major α-haloketones components. For example, with a mixture of starting materials such as 70 percent 1,3-dihaloacetones and 30 percent 1-haloacetone, the product will contain substantially 1,3-dihalo-2-propanol.

The following examples are for illustrative purposes only and should not be taken as limiting the scope of either the present Specification or the claims. Unless otherwise stated, all parts and percentages are by weight.

General Experimental Procedures

Catalyst Synthesis: Catalysts are prepared by impregnating silica with aqueous metal salt solutions of IrCl₃.3H₂O and RuCl₃.H₂O. The mixed metal systems is prepared by coimpregnation the two metal salts into silica or by impregnation of one metal salt (and dried) followed by integration of the other metal salt. The catalysts are air dried and then prereduced under dynamic H₂/N₂ (5% hydrogen) at 473° K (200° C.). The catalysts are then stored and handled in air.

Reactor System A: The reactor consists of a 6.35E-3 m×3.05E-1 m (0.25 inch×12 inches) Hastelloy tube wrapped with heat tape and insulation, a liquid pump, and two flow controllers capable of delivering 3.55E6 Pa (500 pounds per square inch gauge (psig)) of hydrogen and nitrogen. The feed mixture of gas and liquid feed enters the reactor at the bottom and exits the reactor at the top; and then the feed mixture passes through a back pressure regulator to an ambient pressure sample system; and then to a caustic scrubber.

Reactor A Operation: Catalyst is loaded into the reactor by removing the outlet line of the depressurized reactor and adding 7.5E-7 m³ of Sigma glass beads (425-600 microns, acid-washed) and then 1E-3 kg of catalyst is added to the tube and another 7.5E-7 m³ of glass beads is added to the reactor. The outlet line is connected and the reactor is purged with nitrogen at ambient pressure for one hour and the reactor is heated to 358 K (85° C.). The reactor is then filled with hydrogen to a pressure of 3.55E6 Pa (500 psig) and after ½ hour the liquid feed is started.

Reactor System B Description and Operation: A 300 mL Hastelloy C Parr reactor vessel is used in this case. The reactor is loaded with a catalyst charge and the reactor vessel is evacuated and nitrogen flushed three times. A solvent/α-haloketone mixture is sparge degassed with nitrogen and added to the Parr reactor with a syringe. The reactor is pressurized/vented to 250/20 psig (1.8 mPa/241 kPa) nitrogen and 100/20 psig (793 kPa/241 kPa) hydrogen, then placed under 100 psig (793 kPa) hydrogen and heated to 35° C. Samples were removed by syringe after venting the reactor to less than 15 psig (207 kPa).

Analysis: Samples are analyzed by gas chromatography (GC) using a Hewlett Packard HP-6890 gas chromatograph equipped with a 30 m Rtx-5 capillary column with split injection. Approximately 120 μL of the reaction mixture is dissolved in 5E-6 m³ (5 mL) of o-dichlorobenzene which contained a known amount of chlorobenzene as a GC standard (typically 0.05 weight percent). “Selectivity” is defined as the ratio of α-haloalcohol over the combined products formed.

### EXAMPLE 1

Example 1 demonstrates the performance of an 8% Ir/2% Ru/Silica catalyst for the hydrogenation of 1,3-dichloroacetone.

In Reactor A, 1.0 g of an 8.0% Ir/2.0% Ru/silica catalyst is loaded in a reactor as described above under the General Experimental Procedures. A liquid feed consisting of a 10.2 wt % 1,3 dichloroacetone/dioxane mixture is prepared and sparged with nitrogen. The feed rate is 2.2E-9 m³/s (0.132 cc/minute) which corresponds to a contact time of 4,440 seconds as described earlier. As described in the General Experimental Procedures, 85° C. and 500 psig (3.55E6 Pa) H₂ are the standard reaction conditions. The reaction is sampled periodicaly over 80.5 hours and analyzed. The results of the analysis are shown in the following Table I where “selectivity” is defined as the ratio of 1,3-dichloro-2-propanol over the combined products formed.

TABLE I
GC Analysis

| Time (minutes) | % Conversion | % Selectivity |
|---|---|---|
| 1.5000 | 100.00 | 95.000 |
| 5.2500 | 100.00 | 94.000 |
| 21.420 | 100.00 | 94.000 |
| 28.000 | 100.00 | 94.000 |
| 47.000 | 100.00 | 94.000 |
| 80.500 | 99.000 | 93.000 |

### Example 2

Example 2 demonstrates the performance of an 8% Ir/2% Ru/Silica catalyst for the hydrogenation of 1-chloroacetone.

In Reactor A, 1.0 g of an 8.0% Ir/2.0% Ru/silica catalyst is loaded in a reactor as described above in the General Experimental Procedures. A liquid feed consisting of a 7.1 wt % 1-chloroacetone/dioxane mixture is prepared and sparged with nitrogen. The feed rate is 3.0E-9 m³/s (0.182 cc/minute) which corresponds to a contact time of 4,675 seconds. As described in the General Experimental Procedures, 85° C. and 500 psig (3.55E6 Pa) H₂ are the standard reaction conditions. The reaction is sampled periodicaly over 68.25 hours and analyzed. The results of the analysis are shown in the following Table II where “selectivity” is defined as the ratio of 1-chloro-2-propanol over the combined products formed.

TABLE II
GC Analysis

| Time (minutes) | % Conversion | % Selectivity |
|---|---|---|
| 2.2500 | 100.00 | 95.000 |
| 4.2500 | 100.00 | 95.000 |
| 21.330 | 100.00 | 95.000 |
| 26.000 | 100.00 | 93.000 |
| 51.000 | 100.00 | 93.000 |
| 68.250 | 100.00 | 94.000 |

### Example 3

Example 3 provides a comparison of a heterogeneous platinum (Pt) oxide catalyst (Adams' catalyst) with an 8% Ir/2% Ru/silica catalyst. Adams' catalyst has previously been disclosed in U.S. Pat. No. 3,189,656 for the hydrogenation of 1,3-dichloro-1,1,3,3-tetraflouroacetone to 1,3-dichloro-1,1,3,3-tetraflouro-2-propanol.

Reactor B is loaded with either 0.025 g Adams' catalyst or 0.25 g 8% Ir/2% Ru/silica and the reactor vessel is evacuated and nitrogen flushed three times. 1,3-dichloroacetone (2.5 g) dissolved in 1,4-dioxane (50 mL) is sparge degassed with nitrogen and added to the Parr reactor with a syringe. The reactor is pressurized/vented to 250/20 psig (1.8 mPa/241 kPa) nitrogen and 100/20 psig (793 kPa/241 kPa) hydrogen, then placed under 100 psig (690 kPa) hydrogen and heated to 35° C. After 8 hours of reaction, a sample is removed by syringe and analyzed by GC after venting the reactor to less than 15 psig (207 kPa). The results of the analysis of this Example 3 is shown in Table III below. The results in Table III demonstrate the Adams' catalyst is inferior compared to the 8% Ir/2% Ru/silica catalyst of the present invention for the hydrogenation of non-fluorine containing α-haloketones such as 1,3-dichloroacetone.

TABLE III
GC Analysis

| | | | % Selectivity |
|---|---|---|---|
| | % Conversion | % Yield | to |
| | 1,3-Dichloro | 1,3-Dichloro- | 1,3-Dichloro- |
| Catalyst | acetone | 2-propanol | 2-propanol |
| Comparative | 38.7% | 3.0% | 7.8% |
| Example A: Pt | | | |
| oxide (Adams' | | | |
| Catalyst) | | | |
| 8% Ir/2% | 16.2% | 11.6% | 71.6% |
| Ru/Silica | | | |

What is claimed is:

1. A process to make an α-haloalcohol comprising the step of reacting one or more α-haloketones of the following general Formula I: 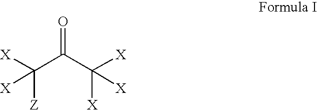
wherein each “X” is independently a halogen atom excluding fluorine, a hydrogen atom, or an organic group; and “Z” is a halogen atom excluding fluorine; with a hydrogenating agent in the presence of a transition metal-containing heterogeneous catalyst, under conditions such that an α-haloalcohol of the following general Formula II is formed: 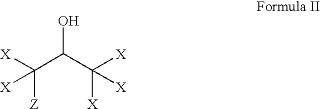
wherein each “X” is independently a halogen atom excluding fluorine, a hydrogen atom, or an organic group; and “Z” is a halogen atom excluding fluorine.

2. The process described in claim 1 wherein the α-haloketone is selected from the group comprising 1,3-dichloroacetone; 1,3-dibromoacetone; 1-bromo-3-chloroacetone; 1-chloroacetone, 1-bromoacetone, or mixtures thereof.

3. The process described in claim 1 wherein the α-haloketone is an unsubstituted 1,3-dichloroacetone and the α-haloalcohol formed is 1,3-dichloro-2-propanol.

4. The process described in claim 1 wherein the α-haloketone is an unsubstituted 1-chloroacetone and the α-haloalcohol formed is 1-chloro-2-propanol.

5. The process as described in claim 1 wherein the hydrogenating agent is molecular hydrogen.

6. The process as described in claim 5 wherein the ratio of molecular hydrogen to α-haloketone is at least about 0.75:1.

7. The process as described in claim 5 wherein the ratio of molecular hydrogen to α-haloketone is at least about 0.6:1.

8. The process as described in claim 1 wherein the catalyst comprises a Group VIIIA metal.

9. The process as described in claim 8 wherein the catalyst comprises iridium, ruthenium, and mixtures thereof.

10. The process in claim 9 wherein the catalyst comprises a mixture of iridium metal and ruthenium metal in an atomic ratio of iridium metal to ruthenium metal of from about 0.02 to about 15.

11. The process of claim 10 wherein the atomic ratio of iridium metal to ruthenium metal is from about 0.15 to 8.

12. The process of claim 11 wherein the atomic ratio of iridium metal to ruthenium metal is from about 0.3 to 2.

13. The process as described in claim 1 wherein the catalyst includes a Group I or transition metal promoter ion.

14. The process as described in claim 13 wherein the promoter ion is selected from the group consisting essentially of Li, Na, K, Cs, Mo, W, V, Re, Mn, and mixtures thereof.

15. The process as described in claim 1 wherein the catalyst further contains a coordinating ligand.

16. The process as described in claim 15 wherein the ligand is selected from the group consisting essentially of phosphines, 1,5-cycloocctadiene (COD), arsines, stibines, carbon monoxide, ethers, cyclopentadienyl, sulfoxides, aromatic amines, and mixtures thereof.

17. The process as described in claim 16 wherein the ligand is phosphine.

18. The process of claim 1 wherein the heterogeneous catalyst support is selected from the group consisting essentially of carbon, silica, alumina, titania, zirconia, cross-linked polystyrene, and combinations thereof.

19. The process of claim 1 wherein the heterogeneous catalyst is in the form of a heterogeneous catalyst bed in a reactor, and wherein the heterogeneous catalyst is present in the reaction mixture at a ratio of about 0.0001 to about 100 moles of catalyst metal for each mole of α-haloalcohol which passes through the bed per hour.

20. The process as described in claim 1 which is carried out at a temperature of about 0° C. to about 200° C.

21. The process as described in claim 1 which is carried out with a molecular hydrogen partial pressure of at least about 14 psia.

22. The process as described in claim 1 wherein the reaction mixture further comprises a solvent.

23. The process of claim 22 wherein the solvent is selected from the group consisting essentially of aromatic hydrocarbons, aliphatic hydrocarbons, chlorinated hydrocarbons, ethers, glymes, glycol ethers, esters, alcohols, amides, water, and mixtures thereof.

24. The process as described in claim 22 wherein the solvent is present in the reaction mixture in an amount from about 0 to 99.99 weight percent.

25. The process of claim 1 wherein the reaction mixture further comprises an acid scavenger.

26. The process of claim 25 wherein the acid scavenger is selected from the group consisting essentially of alkali metal carbonates; alkali metal bicarbonates; alkali metal carboxylates; ammonium and phosponium carboxylates, bicarbonates, and carbonates; epoxides and mixtures thereof.

27. The process of claim 25 wherein the acid scavenger is epichlorohydrin.

28. The process as described in claim 1 comprising the step of contacting an α-haloketone with at least a stoichiometric quantity of molecular hydrogen in the presence of a ruthenium-containing catalyst, an iridium-containing catalyst, or a mixed iridium ruthenium-containing catalyst and a solvent.

29. A process to make epoxides comprising the steps of:
(a) reducing an α-haloketone as described in claim 1 to form an α-haloalcohol; and
(b) contacting the α-haloalcohol with a base to form an epoxide.

30. A process to make epoxides comprising the steps of:
(a) α-halogenating a ketone to make an α-haloketone;
(b) reducing the α-haloketone as described in claim 1 to form an α-haloalcohol; and
(c) contacting the α-haloalcohol with a base to form an epoxide.

31. The process of claim 29 or claim 30 wherein the α-haloketone is a mixture of one or more α-haloketones and the α-haloalcohol is a mixture of one or more α-haloalcohols.

32. A process to make epihalohydrin comprising the steps of:
(a) reducing 1,3-dihaloacetone as described in claim 1 to form 1,3-dihalo-2-propanol; and
(b) contacting the 1,3-dihalo-2-propanol with a base to form an epihalohydrin.

33. A process to make epihalohydrin comprising the steps of:
(a) α-halogenating acetone to make 1,3-dihaloacetone;
(b) reducing 1,3-dihaloacetone as described in claim 1 to form 1,3-dihalo-2-propanol; and
(c) contacting the 1,3-dihalo-2-propanol with a base to form an epihalohydrin.

34. The process of claim 32 or claim 33 wherein the 1,3-dihaloacetone is in a mixture with other ketones; wherein the mixture contains predominantly 1,3-dihaloacetone; and wherein the product formed is predominantly epihalohydrin.

35. A process to make propylene oxide comprising the steps of:
(a) reducing 1-haloacetone as described in claim 1 to form 1-halo-2-propanol; and
(b) contacting the 1-halo-2-propanol with a base to form propylene oxide.

36. A process to make propylene oxide comprising the steps of:
(a) α-halogenating acetone to make 1-haloacetone;
(b) reducing 1-haloacetone as described in claim 1 to form 1-halo-2-propanol; and
(c) contacting the 1-halo-2-propanol with a base to form propylene oxide.

37. The process of claim 35 or claim 36 wherein the 1-haloacetone is in a mixture with other ketones; wherein the mixture contains predominantly 1-haloacetone; and wherein the product formed is predominantly propylene oxide.
